(19) United States
(12) Patent Application Publication
(10) Pub. No.: US 2026/0069185 A1
(43) Pub. Date: Mar. 12, 2026

(54) ELECTROCARDIOGRAM (EKG) DEVICE FOR AN AUTOMOTIVE STEERING WHEEL AND AEROBIC EXERCISE EQUIPMENT

(71) Applicant: Leo Polosajian, Reseda, CA (US)

(72) Inventor: Leo Polosajian, Reseda, CA (US)

(21) Appl. No.: 18/830,225

(22) Filed: Sep. 10, 2024

(51) Int. Cl. A61B 5/26; A61B 5/00; A61B 5/28; A61B 5/361

(52) U.S. Cl. CPC A61B 5/26; A61B 5/28; A61B 5/361; A61B 5/6895; A61B 5/7264; A61B 5/742; A61B 5/746; A61B 2560/0443; A61B 2560/0468

(57) ABSTRACT

A system for measuring an electrocardiogram signal of a driver of a vehicle or a user of aerobic exercise equipment includes an electrocardiogram device on the steering wheel or the aerobic exercise equipment configured to measure the electrocardiogram signal. The electrocardiogram device includes a first electrode; a second electrode spaced apart from the first electrode; a nonvolatile memory device coupled to at least one of the first electrode or the second electrode; a processor coupled to at least one of the first electrode or the second electrode; and a network adapter. The nonvolatile memory device includes computer-readable instructions which, when executed by the processor, cause the electrocardiogram device to measure the electrocardiogram signal with the first electrode and the second electrode and to transmit the electrocardiogram signal from the network adapter to an external electronic device.

### BACKGROUND

### 1. Field

The present disclosure relates to electrocardiogram (EKG) devices for automotive steering wheels and aerobic exercise equipment.

### 2. Description of the Related Art

Atrial fibrillation (AFib) is a medical condition in which an individual experiences an irregular and often very rapid heart rhythm. Atrial fibrillation can lead to blood clots in the heart and increase the patient's risk of stroke, heart failure, and other heart-related complications. However, individuals may only rarely measure their electrocardiogram signal, such as during annual checkups. Accordingly, atrial fibrillation may go undetected before it causes serious health complications.

The above-described information disclosed in this section is only for improving the understanding of the background of the present disclosure and thus it may include information that does not constitute prior art.

### SUMMARY

The present disclosure relates to various embodiments of systems for measuring an electrocardiogram signal of an individual. In one embodiment, the system includes a device such as an aerobic exercise equipment or a steering wheel of a vehicle; and an electrocardiogram device on the device configured to measure the electrocardiogram signal of the individual. The electrocardiogram device includes a first electrode; a second electrode spaced apart from the first electrode; a nonvolatile memory device coupled to at least one of the first electrode or the second electrode; a processor coupled to at least one of the first electrode or the second electrode; and a network adapter. The nonvolatile memory device includes computer-readable instructions which, when executed by the processor, cause the electrocardiogram device to measure the electrocardiogram signal with the first electrode and the second electrode and to transmit the electrocardiogram signal from the network adapter to an external electronic device.

Each of the first electrode and the second electrode may be curved.

Each of the first and second electrodes may be curved along a circumferential direction of the steering wheel.

Each of the first electrode and the second electrode may be further curved along a radial direction of the steering wheel.

The first electrode may be located at a first angle in a range from approximately 15 degrees to approximately 75 degrees in a counterclockwise direction with respect to a top-dead-center position of the steering wheel, and the second electrode may be located at a second angle in a range from approximately 15 degrees to approximately 75 degrees in a clockwise direction with respect to the top-dead-center position of the steering wheel.

Each of the first electrode and the second electrode may include at least one finger recess configured to accommodate at least a portion of one or more of the individual's fingers.

The system may further include a portable electronic device in communication with the electrocardiogram device and the vehicle.

The computer-readable instructions, when executed by the processor, may cause the electrocardiogram device to compare the electrocardiogram signal to a normal electrocardiogram signal.

The computer-readable instructions, when executed by the processor, may cause the electrocardiogram device to transmit a signal to the portable electronic device, the signal being configured to cause a display of the portable electronic device to display an alert.

The alert may include an indication of atrial fibrillation.

The electrocardiogram device may be integral with the steering wheel of the vehicle.

The electrocardiogram device may be removable from the steering wheel of the vehicle.

The system may further include a fastener or an adhesive detachably coupling the first electrode and the second electrode to the steering wheel of the vehicle.

The present disclosure also relates to various methods of retrofitting a device to measure electrocardiogram signals of an individual. In one embodiment, the method includes attaching a first electrocardiogram electrode and a second electrocardiogram electrode to the device (an aerobic exercise equipment or a steering wheel of a vehicle); electrically pairing the first electrocardiogram electrode and the second electrocardiogram electrode to a portable electronic device; and measuring, from the first electrocardiogram electrode and the second electrocardiogram electrode, the electrocardiogram signals of the individual in response to the individual placing the individual's hands on the first electrocardiogram electrode and the second electrocardiogram electrode; and wirelessly transmitting the electrocardiogram signal to the portable electrode device during driving of vehicle.

The method may also include displaying, on a display of the portable electronic device, a graphic representation of the electrocardiogram signal.

The method may also include comparing the electrocardiogram signal to a normal electrocardiogram signal.

The method may include displaying, on the display of the portable electronic device, an alert in response to the electrocardiogram signal being different than the normal electrocardiogram signal.

The alert may include an indication of atrial fibrillation.

The portable electronic device may be a mobile phone.

The method may also include wirelessly transmitting the electrocardiogram signal to the vehicle; and displaying, on a display of the vehicle, an alert in response to the electrocardiogram signal.

This summary is provided to introduce a selection of features and concepts of embodiments of the present disclosure that are further described below in the detailed description. This summary is not intended to identify key or essential features of the claimed subject matter, nor is it intended to be used in limiting the scope of the claimed subject matter. One or more of the described features may be combined with one or more other described features to provide a workable device.

### BRIEF DESCRIPTION OF THE DRAWINGS

These and other features and advantages of embodiments of the present disclosure will become more apparent by reference to the following detailed description when considered in conjunction with the following drawings. In the drawings, like reference numerals are used throughout the figures to reference like features and components. The figures are not necessarily drawn to scale.

FIGS. 1A-1B are a perspective view and a schematic block diagram, respectively, of an electrocardiogram (EKG) system in an automotive vehicle according to one embodiment of the present disclosure;

FIGS. 2A-2B are a perspective view and a schematic block diagram, respectively, of electrocardiogram (EKG) system on an aerobic workout machine according to one embodiment of the present disclosure; and

FIG. 3 is a flowchart illustrating tasks of a method of retrofitting a device to include an electrocardiogram (EKG) system according to one embodiment of the present disclosure.

### DETAILED DESCRIPTION

The present disclosure relates to various embodiments of an electrocardiogram (EKG) system for measuring an electrocardiogram signal of a driver of a vehicle or the user of aerobic exercise equipment. The system includes a pair of electrodes that are either integral with or detachable from the steering wheel of the vehicle or the handles (or hand grips) of the exercise equipment. The electrocardiogram signal measured by the EKG system may be utilized to detect the incidence of atrial fibrillation, which increases the risk of ischemic stroke.

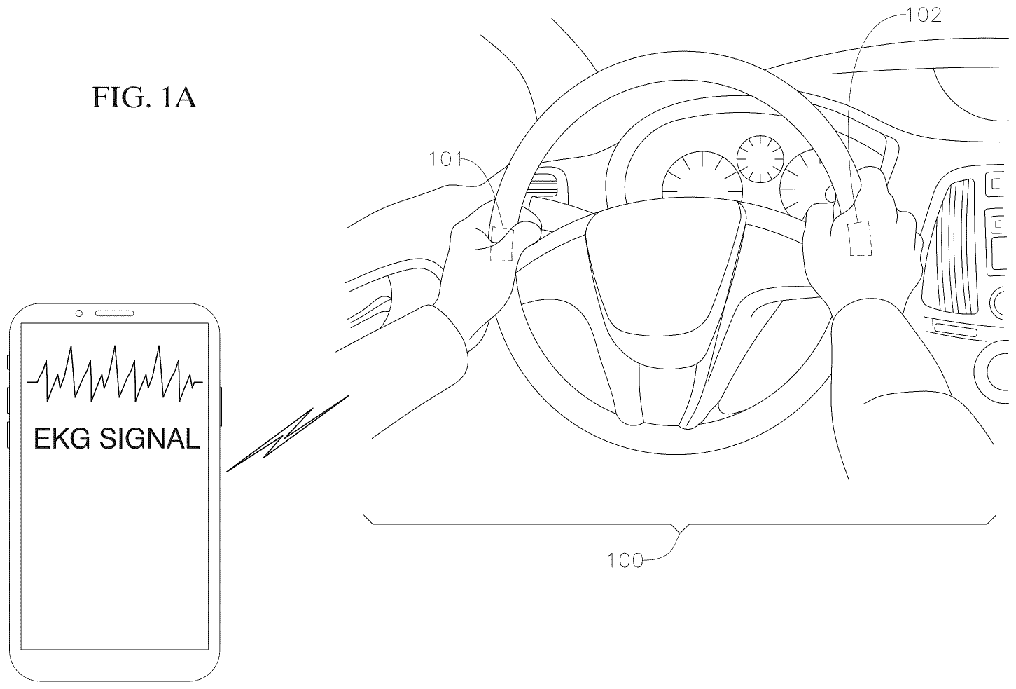

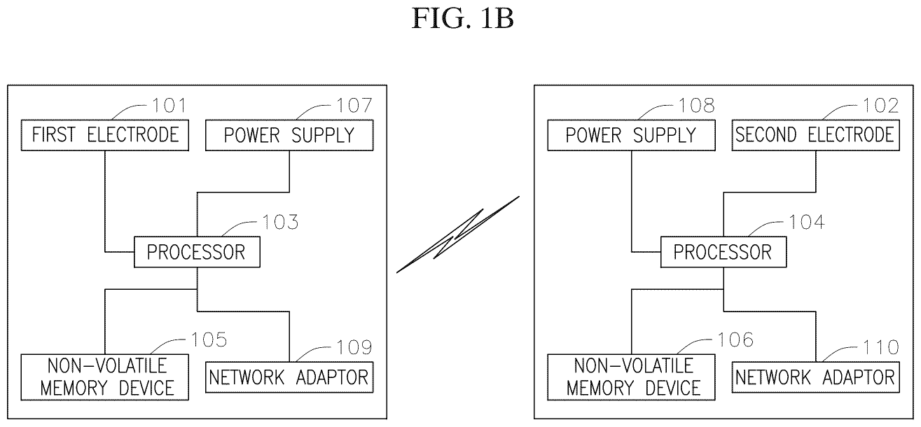

FIGS. 1A-1B depict an electrocardiogram (EKG) system 100 according to one embodiment of the present disclosure that is configured to be connected to the steering wheel of a vehicle. In the illustrated embodiment, the EKG system 100 includes a pair of electrodes 101, 102 (e.g., a left electrode 101 and a right electrode 102), a pair of processors 103, 104 coupled to the pair of electrodes 101, 102, respectively, and a pair of non-volatile memory devices 105, 106 (e.g., read-only memory (“ROM”), programmable ROM (“PROM”), erasable programmable ROM (“EPROM”), electrically erasable programmable ROM “EEPROM”), flash memory, etc.) coupled pair of processors 103, 104, respectively. In one or more embodiments, the EKG system 100 may also include a pair of power supplies 107, 108 (e.g., one or more secondary batteries) coupled to the processors 103, 104, respectively. In one or more embodiments, the EKG system 100 may not include the power supplies 107, 108 and the EKG system 100 may be connected to the battery of the vehicle. Additionally, in one or more embodiments, the EKG system 100 may include a pair of network adapters 109, 110 (e.g., a WiFi chip and/or a Bluetooth™ chip) coupled to the processors 103, 104, respectively. The network adapters 109, 110 are configured to enable the EKG system 100 to wirelessly communicate with one or more electronic devices, such as a mobile electronic device (e.g., a smartphone or a tablet computer). The network adapters 109, 110 may also be configured to enable In one or more embodiments, the EKG system 100 may not include the network adapter 106 and the EKG system 100 may be wired (e.g., via a detachable cable or hardwired) to another electronic device, such as a mobile electronic device (e.g., a smartphone or a tablet computer) and/or to a display (e.g., an LED or LCD screen) integrated into the vehicle.

Although in one or more embodiments the EKG system 100 may include one processor 103, 104 for each of the electrodes 101, 102 (e.g., a first processor connected 103 to the first electrode 101 and a second processor 104 connected to the second electrode 102), in one or more embodiments the pair of electrodes 101, 102 may share a single processor 103. Additionally, although in one or more embodiments the EKG system 100 may include one non-volatile memory device 105, 106 for each of the electrodes 101, 102 (e.g., a first non-volatile memory device 105 connected to the first electrode 101 and a second non-volatile memory device 106 connected to the second electrode 102), in one or more embodiments the pair of electrodes 101, 102 may share a single non-volatile memory device.

The non-volatile memory devices 105, 106 include computer-readable instructions (i.e., computer-executable instructions) which, when executed by the processor 103, 104, cause the EKG system 100 to perform various functions. For instance, in one or more embodiments, the computer-readable instructions, when executed by the processor 103, 104, cause the pair of electrodes 101, 102 to detect the presence of the driver's hands or fingers on the pair of electrodes 101, 102 and to measure an electrocardiogram signal of the driver (e.g., the driver's heart rhythm). The electrocardiogram signal is a measure of heart's electrical activity over repeated cardiac cycles and is measured in voltage as a function of time. The electrocardiogram signal includes three main components: (i) the P-wave, which represents the depolarization of the atria of the heart; (ii) the QRS complex, which represents depolarization of the ventricles of the heart; and (iii) the T-wave, which represents the repolarization of the ventricles of the heart.

Additionally, in one or more embodiments, the computer-readable instructions, when executed by the processor 103, 104, cause the processor 103, 104 to compare the measured electrocardiogram signal against a baseline (i.e., normal) cardiogram waveform and/or to analyze the electrocardiogram signal for abnormalities. For instance, in one or more embodiments, the computer-readable instructions, when executed by the processor 103, 104, cause the processor 103, 104 to analyze the measured electrocardiogram signal for one or more different abnormalities, such ST-segment abnormalities, T-wave abnormalities, or both ST-T abnormalities. These abnormalities may be utilized to determine a variety of different health conditions in the driver, such as a previous heart attack (myocardial infarction), an abnormal heart rhythm (arrhythmia), an inadequate supply of blood and oxygen to the heart (ischemia), and/or an excessive thickening (hypertrophy) of the heart's muscular walls. In one or more embodiments, the baseline waveform and/or the abnormality parameters may be stored locally in the non-volatile memory device of the EKG system 100 or they may be stored in a remote electronic device (e.g., a server) accessible by the network adapter 109, 110 of the EKG system 100.

In one or more embodiments, the computer-readable instructions, when executed by the processor 103, 104, cause the EKG system 100 to transmit, via the network adapter 106, a signal to the remote electronic device (e.g., a mobile phone or a tablet computer in the vehicle). In one or more embodiments, the signal is configured to cause a display of the remote electronic device to display the electrocardiogram signal generated by the electrodes 101, 102, and/or information regarding, related to, derived from, or inferred from the electrocardiogram signal, such as a summary of the electrocardiogram signal (e.g., the driver's heart rate and/or rhythm) and/or a medical diagnosis determined from the electrocardiogram signal (e.g., atrial fibrillation). In one or more embodiments, the signal transmitted from the EKG system to the remote electronic device is configured to cause the remote electronic device to generate an alert in response to the analysis of the electrocardiogram signal indicating an abnormality in the driver's heart rhythm. In one or more embodiments, the alert may be a visual alert (e.g., a graphic and/or text-based alert) displayed on the display of the remote electronic device, an auditory alert emitted by a speaker of the remote electronic device, and/or a haptic alert generated by the remote electronic device. Additionally, in one or more embodiments, the signal may cause the remote electronic device to store, in local memory and/or in a cloud computing device, the electrocardiogram signal, the information relating to the electrocardiogram signal (e.g., the summary and/or diagnosis), and/or the alert.

Additionally, in one or more embodiments, the pair of electrodes 101, 102 may be integral with the steering wheel of the vehicle or the pair of electrodes 101, 102 may be removable from the steering wheel. For instance, in one or more embodiments, the electrodes 101, 102 may be detachably coupled to the steering wheel with an adhesive and/or fasteners (e.g., hook-and-loop fasteners).

In one or more embodiments, each of the electrodes 101, 102 may be a thin plate. In one or more embodiments, the electrodes 101, 102 may be generally square or rectangular. In one or more embodiments, the configuration of the electrodes 101, 102 may conform or substantially conform to a portion of the steering wheel in the vehicle. The thin plate may be arcuate (e.g., curved in a circumferential direction of the steering wheel). In one or more embodiments, the thin plate may curved in the radial direction of the steering wheel. In one or more embodiments, the thin plate of each of the electrodes 101, 102 may be curved in both the circumferential direction and the radial direction of the steering wheel.

In one or more embodiments, the first electrode 101 may be located at a first angle θ₁ in a range from approximately 15 degrees to approximately 75 degrees in a counterclockwise direction with respect to a top-dead-center position of the steering wheel, and the second electrode 102 may located at a second angle θ₂ in a range from approximately 15 degrees to approximately 75 degrees in a clockwise direction with respect to the top-dead-center position of the steering wheel. In one or more embodiments, the first and second electrodes 101, 102 may be symmetric or substantially symmetric about the top-dead-center position of the steering wheel.

In one or more embodiment, each of the first electrode 101 and the second electrode 102 may each include at least one finger recess (e.g., a depression) configured to accommodate at least a portion of one or more of the driver's fingers. In one or more embodiments, the first and second electrodes 101, 102 may not include a finger recess.

The term “processor” is used herein to include any combination of hardware, firmware, and software, employed to process data or digital signals. The hardware of a processor may include, for example, application specific integrated circuits (ASICs), general purpose or special purpose central processors (CPUs), digital signal processors (DSPs), graphics processors (GPUs), and programmable logic devices such as field programmable gate arrays (FPGAs). In a processor, as used herein, each function is performed either by hardware configured, i.e., hard-wired, to perform that function, or by more general purpose hardware, such as a CPU, configured to execute instructions stored in a non-transitory storage medium. A processor may be fabricated on a single printed wiring board (PWB) or distributed over several interconnected PWBs. A processor may contain other processors; for example, a processor may include two processors, an FPGA and a CPU, interconnected on a PWB.

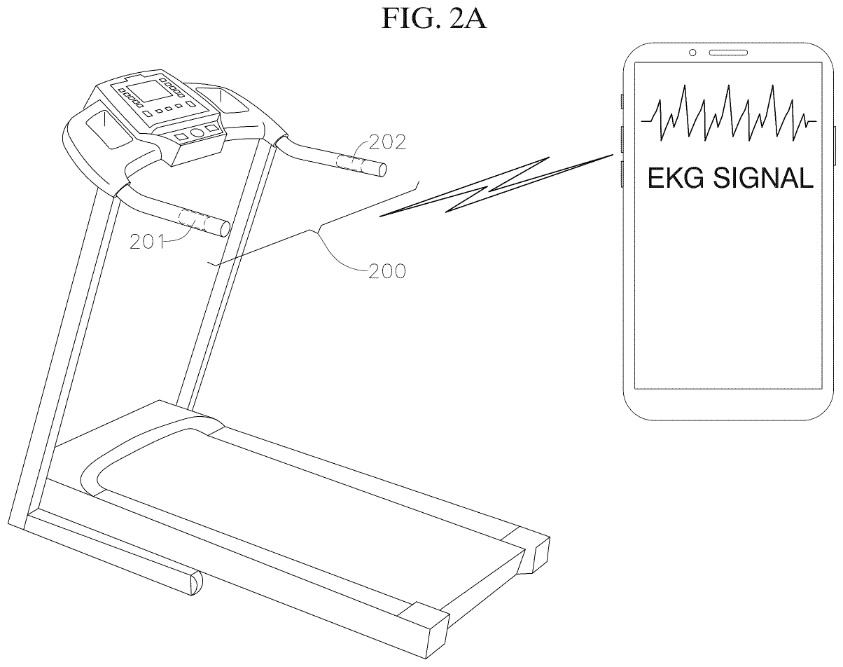

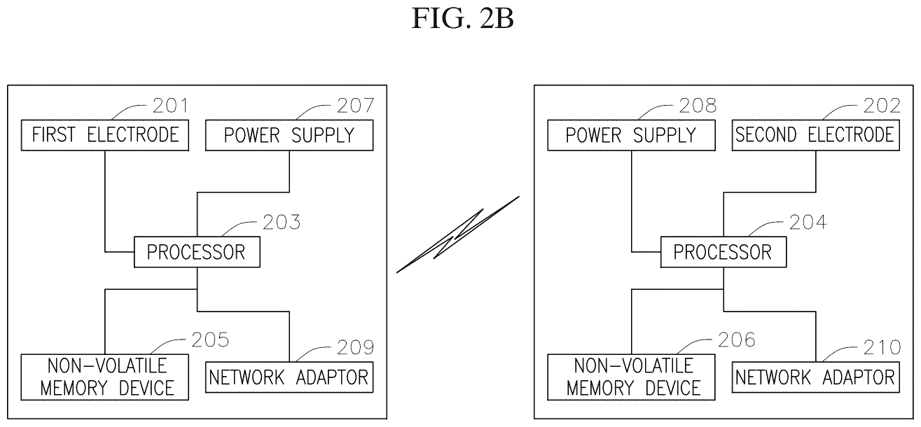

FIGS. 2A-2B depicts an electrocardiogram (EKG) system 200 according to one embodiment of the present disclosure that is configured to be connected to an aerobic exercise equipment, such as a stationary bike, an elliptical machine, a stair climbing device, or a treadmill. In the illustrated embodiment, the EKG system 200 includes a pair of electrodes 201, 202, a pair of processors 203, 204 coupled to the pair of electrodes 201, 202, respectively, and a pair of non-volatile memory devices 205, 206 coupled to the processors 203, 204, respectively. In one or more embodiments, the EKG system 200 may also include at least one power supply 205 (e.g., one or more secondary batteries) coupled to the processor 203. In one or more embodiments, the EKG system 200 may not include a power supply 205 and the EKG system 200 may be connected to the power supply of the aerobic exercise equipment. Additionally, in one or more embodiments, the EKG system 200 may include at least one network adapter 206 (e.g., a WiFi chip and/or a Bluetooth™ chip). The network adapter 206 is configured to enable the EKG system 200 to wirelessly communicate with one or more electronic devices, such as a mobile electronic device (e.g., a smartphone or a tablet computer). In one or more embodiments, the EKG system 200 may not include the network adapter 206 and the EKG system 200 may be wired (e.g., via a detachable cable or hardwired) to another electronic device, such as a mobile electronic device (e.g., a smartphone or a tablet computer) and/or to a display (e.g., an LED or LCD screen) integrated into the aerobic exercise equipment.

Although in one or more embodiments, the pair of electrodes 201, 202 may share a single processor 203, in one or more embodiments the EKG system 200 may include one processor for each of the electrodes 201, 202 (e.g., a first processor connected to the first electrode 201 and a second processor connected to the second electrode 202). Additionally, although in one or more embodiments, the pair of electrodes 201, 202 may share a single non-volatile memory device 204, in one or more embodiments the EKG system 200 may include one non-volatile memory device 204 for each of the electrodes 201, 202 (e.g., a first non-volatile memory device connected to the first electrode 201 and a second non-volatile memory device connected to the second electrode 202).

The non-volatile memory device 204 includes computer-readable instructions (i.e., computer-executable instructions) which, when executed by the processor 203, cause the EKG system 200 to perform various functions. For instance, in one or more embodiments, the computer-readable instructions, when executed by the processor 203, cause the pair of electrodes 201, 202 to detect the presence of the driver's hands or fingers on the pair of electrodes 201, 202 and to measure an electrocardiogram signal of the user (e.g., the user's heart rhythm). The electrocardiogram signal is a measure of heart's electrical activity over repeated cardiac cycles and is measured in voltage as a function of time. The electrocardiogram signal includes three main components: (i) the P-wave, which represents the depolarization of the atria of the heart; (ii) the QRS complex, which represents depolarization of the ventricles of the heart; and (iii) the T-wave, which represents the repolarization of the ventricles of the heart.

Additionally, in one or more embodiments, the computer-readable instructions, when executed by the processor 203, cause the processor to compare the measured electrocardiogram signal against a baseline (i.e., normal) cardiogram waveform and/or to analyze the electrocardiogram signal for abnormalities. For instance, in one or more embodiments, the computer-readable instructions, when executed by the processor 203, cause the processor 203 to analyze the measured electrocardiogram signal for one or more different abnormalities, such ST-segment abnormalities, T-wave abnormalities, or both ST-T abnormalities. These abnormalities may be utilized to determine a variety of different health conditions in the driver, such as a previous heart attack (myocardial infarction), an abnormal heart rhythm (arrhythmia), an inadequate supply of blood and oxygen to the heart (ischemia), and/or an excessive thickening (hypertrophy) of the heart's muscular walls. In one or more embodiments, the baseline waveform and/or the abnormality parameters may be stored locally in the non-volatile memory device of the EKG system 200 or they may be stored in a remote electronic device (e.g., a server) accessible by the network adapter 206 of the EKG system 200.

In one or more embodiments, the computer-readable instructions, when executed by the processor 203, cause the EKG system 200 to transmit, via the network adapter 206, a signal to the remote electronic device (e.g., a mobile phone or a tablet computer of the user using the aerobic exercise equipment). In one or more embodiments, the signal is configured to cause a display of the remote electronic device to display the electrocardiogram signal generated by the electrodes 201, 202, and/or information regarding, related to, derived from, or inferred from the electrocardiogram signal, such as a summary of the electrocardiogram signal (e.g., the driver's heart rate and/or rhythm) and/or a medical diagnosis determined from the electrocardiogram signal (e.g., atrial fibrillation). In one or more embodiments, the signal transmitted from the EKG system to the remote electronic device is configured to cause the remote electronic device to generate an alert in response to the analysis of the electrocardiogram signal indicating an abnormality in the driver's heart rhythm. In one or more embodiments, the alert may be a visual alert (e.g., a graphic and/or text-based alert) displayed on the display of the remote electronic device, an auditory alert emitted by a speaker of the remote electronic device, and/or a haptic alert generated by the remote electronic device. Additionally, in one or more embodiments, the signal may cause the remote electronic device to store, in local memory and/or in a cloud computing device, the electrocardiogram signal, the information relating to the electrocardiogram signal (e.g., the summary and/or diagnosis), and/or the alert.

Additionally, in one or more embodiments, the pair of electrodes 201, 202 may be integral with the aerobic exercise equipment (e.g., the handles and/or the handgrips of the aerobic exercise equipment) or the pair of electrodes 201, 202 may be removable from the aerobic exercise equipment. For instance, in one or more embodiments, the electrodes 201, 202 may be detachably coupled to the aerobic exercise equipment with an adhesive and/or fasteners (e.g., hook-and-loop fasteners).

In one or more embodiments, each of the electrodes 201, 202 may be a thin plate. In one or more embodiments, the electrodes 201, 202 may be generally square or rectangular. In one or more embodiments, the configuration of the electrodes 201, 202 may conform or substantially conform to a portion of the aerobic exercise equipment (e.g., the handles and/or the handgrips of the aerobic exercise equipment). In one or more embodiments, the thin plate may be curved in the thickness direction of the handles and/or the handgrips of the aerobic exercise equipment.

In one or more embodiment, each of the first electrode 201 and the second electrode 202 may each include at least one finger recess (e.g., a depression) configured to accommodate at least a portion of one or more of the user's fingers. In one or more embodiments, the first and second electrodes 201, 202 may not include a finger recess.

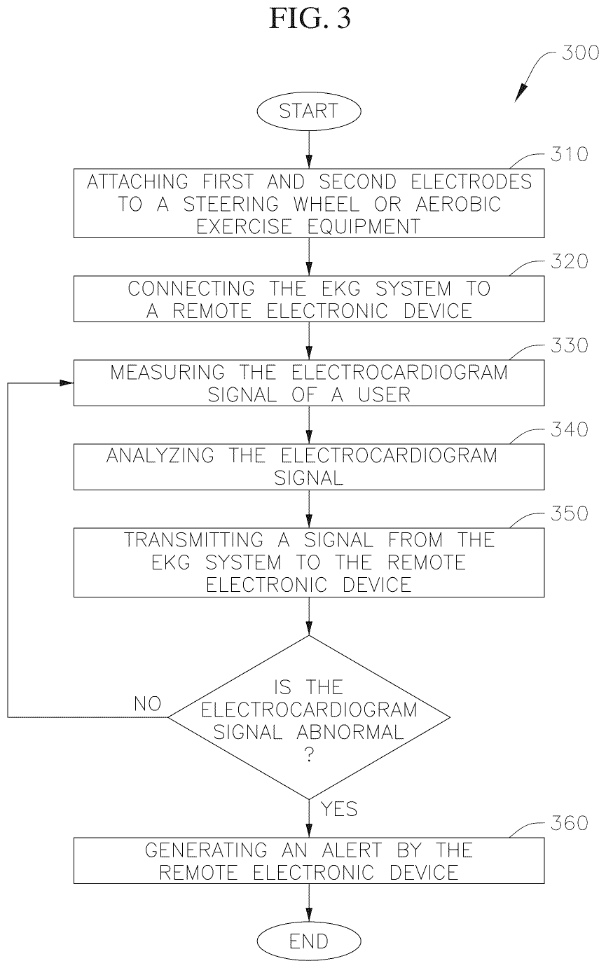

FIG. 3 is a flowchart illustrating tasks of a method 300 of retrofitting a device to include an electrocardiogram (EKG) system according to one embodiment of the present disclosure. The EKG system utilized in the method 300 may be the same as or similar to the EKG system 100 disclosed in FIGS. 1A-1B or the EKG system 200 disclosed in FIGS. 2A-2B.

In the illustrated embodiment, the method 300 includes a task 310 attaching a first electrode and a second electrode to a steering wheel of a vehicle or to handles (or hand grips) of an aerobic exercise equipment, such as a stationary bike, an elliptical machine, a stair climbing device, or a treadmill. The task 310 of attaching the first and second electrodes may include, for example, adhering the first and second electrodes and/or securing the first and second electrodes with one or more fasteners, such as hook-and-loop fasteners.

In the illustrated embodiment, the method 300 also includes a task 320 of connecting the EKG system to a remote electronic device, such as a smartphone or a tablet computer. The task 320 may include wirelessly pairing the EKG system to the remote electronic device. In one or more embodiments, the task 320 may include plugging the remote electronic device into the EKG system.

In the illustrated embodiment, the method 300 also includes a task 330 of measuring the electrocardiogram signal of a user (e.g., the driver of a vehicle or an individual who is exercising) utilizing the first and second electrodes.

In the illustrated embodiment, the method 300 also includes a task 340 of analyzing the electrocardiogram signal (determined in task 330) for abnormalities. In one or more embodiments, the task 340 of analyzing the electrocardiogram signal may include comparing the electrocardiogram signal (determined in task 330) to a baseline (i.e., normal) cardiogram waveform and/or determining the presence and/or absence of waveform abnormalities in the electrocardiogram signal, such as ST-segment abnormalities, T-wave abnormalities, or ST-T abnormalities.

In the illustrated embodiment, the method 300 also includes a task 350 of transmitting a signal from the EKG system to the remote electronic device. The signal contains the electrocardiogram signal and/or information regarding the electrocardiogram signal that was measured or determined in task 330. The signal is configured to cause a display of the remote electronic device to display the electrocardiogram signal and/or information regarding, related to, derived from, or inferred from the electrocardiogram signal, such as a summary of the electrocardiogram signal (e.g., the driver's heart rate and/or rhythm) and/or a medical diagnosis determined from the electrocardiogram signal (e.g., atrial fibrillation).

In the illustrated embodiment, the method 300 also includes a task 360 of generating, by the remote electronic device, an alert in response to the signal transmitted in task 350 indicating that an abnormality was detected in task 340 (i.e., the signal transmitted from the EKG system to the remote electronic device in task 350 is configured to cause the remote electronic device to generate an alert in response to the analysis of the electrocardiogram signal performed in task 340 indicating an abnormality in the driver's heart rhythm). In one or more embodiments, the alert may be a visual alert (e.g., a graphic and/or text-based alert) displayed on the display of the remote electronic device, an auditory alert emitted by a speaker of the remote electronic device, and/or a haptic alert generated by the remote electronic device. In this manner, the method 300 is configured to determine the presence of heart rhythm abnormalities, such as atrial fibrillation, while an individual is driving or working out and can thus enable the individual to seek medical intervention sooner than if the individual waited for an annual checkup.

While this invention has been described in detail with particular references to exemplary embodiments thereof, the exemplary embodiments described herein are not intended to be exhaustive or to limit the scope of the invention to the exact forms disclosed. Persons skilled in the art and technology to which this invention pertains will appreciate that alterations and changes in the described structures and methods of assembly and operation can be practiced without meaningfully departing from the principles, spirit, and scope of this invention, and equivalents thereof.

1. A system for measuring an electrocardiogram signal of an individual, the system comprising:
a device selected from the group consisting of an aerobic exercise equipment or a steering wheel of a vehicle; and
an electrocardiogram device on the device configured to measure the electrocardiogram signal of the individual, the electrocardiogram device comprising:
a first electrode;
a second electrode spaced apart from the first electrode;
a nonvolatile memory device coupled to at least one of the first electrode or the second electrode;
a processor coupled to at least one of the first electrode or the second electrode; and
a network adapter,
wherein the nonvolatile memory device includes computer-readable instructions which, when executed by the processor, cause the electrocardiogram device to measure the electrocardiogram signal with the first electrode and the second electrode and to transmit the electrocardiogram signal from the network adapter to a portable electronic device separate from the aerobic exercise equipment and the vehicle, and
wherein the computer-readable instructions, when executed by the processor, cause the electrocardiogram device to transmit a signal to the portable electronic device, the signal being configured to cause the portable electronic device to store, in a cloud computing device, the electrocardiogram signal.

2. The system of claim 1, wherein the device is an aerobic exercise equipment.

3. The system of claim 1, wherein the device is a steering wheel of a vehicle.

4. The system of claim 1, wherein each of the first electrode and the second electrode is curved.

5. The system of claim 3, wherein each of the first and second electrodes are curved along a circumferential direction of the steering wheel.

6. The system of claim 5, wherein each of the first electrode and the second electrode are further curved along a radial direction of the steering wheel.

7-9. (canceled)

10. The system of claim 1, wherein the computer-readable instructions, when executed by the processor, cause the electrocardiogram device to compare the electrocardiogram signal to a normal electrocardiogram signal.

11. The system of claim 10, wherein the signal transmitted by the electrocardiogram device is configured to cause a display of the portable electronic device to display an alert.

12. The system of claim 11, wherein the alert comprises an indication of atrial fibrillation.

13. The system of claim 1, wherein the electrocardiogram device is integral with the aerobic exercise equipment or the steering wheel of the vehicle.

14. The system of claim 1, wherein the electrocardiogram device is removable from the aerobic exercise equipment or the steering wheel of the vehicle.

15. The system of claim 14, further comprising at least one of a fastener or an adhesive detachably coupling the first electrode and the second electrode to the aerobic exercise equipment or the steering wheel of the vehicle.

16. A method of retrofitting a device to measure electrocardiogram signals of an individual, the method comprising:
attaching a first electrocardiogram electrode and a second electrocardiogram electrode to the device, the device being selected from the group consisting of an aerobic exercise equipment or a steering wheel of a vehicle;
electrically pairing the first electrocardiogram electrode and the second electrocardiogram electrode to a portable electronic device separate from the aerobic exercise equipment and the vehicle; and
measuring, from the first electrocardiogram electrode and the second electrocardiogram electrode, the electrocardiogram signals of the individual in response to the individual placing the individual's hands on the first electrocardiogram electrode and the second electrocardiogram electrode; and
wirelessly transmitting the electrocardiogram signal to the portable electronic device during driving of vehicle or use of the aerobic exercise equipment, the portable electronic device storing the electrocardiogram signal in a cloud computing device.

17. The method of claim 16, displaying, on a display of the portable electronic device, a graphic representation of the electrocardiogram signal.

18. The method of claim 16, further comprising comparing the electrocardiogram signal to a normal electrocardiogram signal.

19. The method of claim 18, displaying, on the display of the portable electronic device, an alert in response to the electrocardiogram signal being different than the normal electrocardiogram signal.

20. The method of claim 19, wherein the alert comprises an indication of atrial fibrillation.

21. The method of claim 16, wherein the portable electronic device is a mobile phone.

22. The method of claim 16, further comprising:
wirelessly transmitting the electrocardiogram signal to the vehicle; and
displaying, on a display of the vehicle, an alert in response to the electrocardiogram signal.
